(19) United States
(12) Patent Application Publication
(10) Pub. No.: US 2023/0258657 A1
(43) Pub. Date: Aug. 17, 2023

(54) COMPOSITION CONTAINING TARC, AND METHOD FOR IMPROVING STORAGE STABILITY OF TARC

(71) Applicant: SEKISUI MEDICAL CO., LTD., Tokyo (JP)

(72) Inventors: Kengo FUJIMURA, Tokyo (JP); Tomoyuki USUI, Tokyo (JP); Yuka SUZUKI, Tokyo (JP); Akiko MATSUMOTO, Tokyo (JP)

(73) Assignee: SEKISUI MEDICAL CO., LTD., Tokyo (JP)

(21) Appl. No.: 18/015,231

(22) PCT Filed: Jul. 9, 2021
(86) PCT No.: PCT/JP2021/025904

§ 371 Date: Jan. 9, 2023

(30) Foreign Application Priority Data
Jul. 10, 2020 (JP) 2020-119026

(51) Int. Cl. G01N 33/68

(52) U.S. Cl. CPC G01N 33/6863

(57) ABSTRACT

An object of the present invention is to provide a TARC-containing composition with high storage stability. Provided is a composition including TARC (Thymus and activation-regulated chemokine) and a polymer having 2-methacryloyloxyethyl phosphorylcholine as a structural unit, and being in liquid form. The aforementioned object is achieved by the composition.

### TECHNICAL FIELD

The present invention relates to a composition containing TARC. Mere specifically, the present invention relates to a composition capable of stably storing TARC for a long period of time. The present invention also relates to a method and kit for measuring TARC, a method for improving the storage stability of TARC, and a TARC adsorption inhibitor.

### BACKGROUND ART

Thymus and activation-regulated chemokine (which may be hereinafter referred to as TARC) is C-C chemokine ligand 17 (CCL17) and is a kind of chemokine having a leukocyte migration activity. TARC attracts Th2 cells, which are one of lymphocytes, to the lesion site to cause IgE production and eosinophil infiltration/activation. It is considered that TARC exacerbates the symptoms of atopic dermatitis by enhancing allergic reactions in this way (Non Patent Literature 1).

Prompt and reliable sedation of atopic dermatitis inflammation is considered important. As compared with other indicators of the disease state of atopic dermatitis such as serum IgE level, peripheral blood eosinophil count, and serum LDH level, TARC matches well with the severity of atopic dermatitis and is considered to reflect the disease state more sensitively (Non Patent Literature 2). Accordingly, use of TARC as a biomarker enables the severity to be grasped objectively and quickly when selecting or changing therapeutic agents for atopic dermatitis, to determine the effects.

A calibration sample needs to be used to quantify a component to be measured in a biological sample. The calibration sample is a sample containing the component to be measured, which is used for applications such as an internal standard or a concentration calibration standard (calibrator). In order to obtain an accurate quantitative value, the calibration sample is required to be stable over time and temperature. The calibration sample is preferably in the form of a fluid solution (which may be hereinafter referred to as “liquid”) for ease of operation. In this case, examples of the matter desired for the calibration sample include maintenance of biological activity (such as antigenicity to specific antibodies, binding activity to specific binding partners of antibodies and lectins, physiological activity of peptide hormones, enzyme activity, and three-dimensional structure as a protein for supporting each activity), prevention of adsorption to containers, and maintenance of antiseptic ability.

As a method for storing a calibration sample in liquid form intended for use in an immunoassay, a method of stabilizing an antigen in coexistence with casein and/or whey protein in a calibration sample (Patent Literature 1), a method of stabilizing insulin in coexistence with a bile acid amide derivative (Patent Literature 2), and a method of stabilizing a soluble interleukin-2 receptor (sIL-2R) in coexistence with a chelating agent (Patent Literature 3) are known. Although it is necessary to study the stabilization method according to the properties of the substance to be stabilized, no detailed studies have been conducted on liquid calibration samples for TARC, at present.

### CITATION LIST

### Patent Literature

Patent Literature 1

Japanese Patent Laid-Open No. 08-005634

Patent Literature 2

Japanese Patent Laid-Open No. 08-012583

Patent Literature 3

Japanese Patent Laid-Open No. 2010-230660

### Non Patent Literature

Non Patent Literature 1

J Allergy Clin Immunol 107: 535-541, 2001

Non Patent Literature 2

Journal of the Japanese Dermatological Association 116 (1): 27-39, 2006

### SUMMARY OF INVENTION

### Technical Problem

It is an object of the present invention to provide a TARC-containing composition with high storage stability.

### Solution to Problem

The present inventors attempted to produce a TARC-containing composition with high storage stability. Then, they nave found that the stability of TARC is improved when TARC is added to a solution containing a polymer having 2-methacryloyloxyethyl phosphorylcholine as a structural unit. The present invention is based on such findings.

Specifically, the present invention is as follows.

<1> A composition including TARC (Thymus and activation-regulated chemokine); and a polymer having 2-methacryloyloxyethyl phosphorylcholine as a structural unit, and being in liquid form.
<2> The composition according to <1>, being a calibration sample solution for measuring TARC.
<3> The composition according to <1> or <2>, being filled in a storage container.
<4> The composition according to <3>, wherein the storage container is plastic or glass.
<5> The composition according to any one of <1> to <4>, wherein the TARC concentration is 10 pg/mL to 1 μg/mL, with respect to the composition.
<6> The composition according to any one of <1> to <5>, wherein a concentration of the polymer is 0.001 mass % to 5 mass % with respect to the composition.
<7> The composition according to any one of <1> to <6>, wherein the polymer has a weight average molecular weight of 1000 to 2000000.
<8> The composition according to any one of <1> to <7>, wherein when the TARC concentration is 500 pg/mL with respect to the composition, the TARC residual rate after storage at 37° C. for 26 days in a plastic container is 80% or more.
<9> The composition according to any one of <1> to <8>, wherein when the TARC concentration is 500 pg/ml, with respect to the composition, the TARC residual rate after storage at 4° C. for 28 days in a plastic container is 80% or more.
<10> The composition according to any one of <1> to <9>, wherein when the TARC concentration is 500 pg/mL with respect to the composition, the TARC residual rate is 30% or more after storage at 10° C. for 12 hours in a glass container.
<11> The composition according to any one of <1> to <10>, wherein the polymer has a weight average molecular weight of 10000 to 70000.
<12> A method for measuring TARC, wherein the method uses the composition according to any one of <1> to <11>.
<13> A kit for measuring TARC, wherein the kit includes the composition according to any one of <1> to <11>.
<14> A method for improving storage stability of TARC, including a step of contacting TARC with a solution containing a polymer having 2-methacryloyloxyethyl phosphorylcholine as a structural unit.
<15> The method for improving the storage stability of TARC according to <14>, wherein the TARC concentration is adjusted so as to be 10 pg/ml, to 1 μg/mL with respect to the solution.
<16> The method for improving the storage stability of TARC according to <14> or <15>, wherein a concentration of the polymer is 0.001 mass % to 5 mass % with respect to the solution.
<17> The method for improving the storage stability of TARC according to any one of <14> to <16>, wherein the polymer has a weight average molecular weight of 10000 to 70000.
<18> An adsorption inhibitor for preventing adsorption of TARC in a solution containing TARC onto a container, including a polymer having 2-methacryloyloxyethyl phosphorylcholine as a structural unit, as active component.
<19> The adsorption inhibitor according to <18>, wherein the TARC concentration in the solution containing TARC is 10 pg/mL to 1 μg/mL with respect to the solution.
<20> The adsorption inhibitor according to <18> or <19>, to be used so that the polymer concentration is 0.001 mass % to 5 mass % with respect to the solution.
<21> The adsorption inhibitor according to any one of <18> to <20>, wherein the polymer has a weight average molecular weight of 10000 to 70000.

### Advantageous Effects of Invention

The present invention can provide a TARC-containing composition with high storage stability, particularly, a TARC-containing calibration sample. Accordingly, the present invention enables TARC in a biological sample to be accurately quantified and the severity of atopic dermatitis to be accurately grasped.

### DESCRIPTION OF EMBODIMENTS

### TARC

In this description, “TARC” means Thymus and activation-regulated chemokine (CCL17). TARC is a kind of chemokine having leukocyte migration activity. TARC has the function to attract Th2 cells, which are one of lymphocytes, to the lesion site to cause IgE production and eosinophil infiltration/activation. Use of TARC as a biomarker enables the severity to be grasped objectively and quickly when selecting or changing therapeutic agents for atopic dermatitis.

TARC can be measured by a known method such as immunological technique. Examples of the immunological technique include ELISA, enzyme immunoassay, surface plasmon resonance, latex agglutination immunoassay (LTIA), chemiluminescence immunoassay, electrochemiluminescence immunoassay, fluorescent antibody method, radioimmunoassay. Western blotting, immunochromatography, and high-performance liquid chromatography (HPLC).

The TARC to be contained in the composition of the present invention may be a commercially available product or one produced or purified by oneself. The TARC to be contained in the composition of the present invention stay be produced in vitro or extracted from the living body.

### Concentration of TARC

The concentration of the TARC to be contained in the composition of the present invention is not limited to the following examples but is preferably 10 pg/mL to 1 μg/mL, more preferably 50 pg/mL to 500 ng/mL, further preferably 100 pg/mL to 100 ng/mL, and most preferably 100 pg/mL to 50 ng/mL, with respect to the composition, in consideration of the stability of TARC.

### Polymer Having 2-methacryloyloxyethyl Phosphorylcholine as Structural Unit

The polymer having 2-methacryloyloxyethyl phosphorylcholine as the structural unit (which may be hereinafter simply referred to as the polymer) in the composition of the present invention is not specifically limited, as long as the structural monomer has 2-methacryloyloxyethyl phosphorylcholine.

The polymer having 2-methacryloyloxyethyl phosphorylcholine as the structural unit may be a homopolymer of 2-methacryloyloxyethyl phosphorylcholine, or a copolymer of 2-methacryloyloxyethyl phosphorylcholine with a further monomer. The further monomer can have a hydrophobic side chain, a hydrophilic side chain, an anionic side chain, a cationic side chain. or a hydrogen-bonding side chain. The further monomer can also have both of a hydrophobic side chain and a hydrophilic side chain.

The constitutional ratio between 2-methacryloyloxyethyl phosphorylcholine and the further monomer may be variable depending on the structure of monomers to be used, but usually a content of 2-methacryloyloxyethyl phosphorylcholine is preferably 5 mol % or more with respect to the copolymer.

The polymer having 2-methacryloyloxyethyl phosphorylcholine as the structural unit may be prepared according to a known synthesis method as described in, for example, Japanese Patent Laid-Open No. 2004-189678. 2-methacryloyloxyethyl phosphorylcholine monomer is sold by, for example, NOF CORPORATION, and those skilled in the art can produce a polymer having required properties by polymerizing with any monomer. Alternatively, for example, LIPIDURE (R) Series sold by NOF CORPORATION may also be used, with LIPIDURE-BL Series sola as a diagnostic agent product being preferable, but is not limited thereto. LIPIDURE-BL Series are polymers in which various monomers are copolymerized with 2-methacryloyloxyethyl phosphorylcholine. LIPIDURE-BL100 Series in which a monomer having a hydrophilic group is copolymerized, LIPIDURE-BL200 Series in which a monomer having a hydrophobic group is copolymerized, LIPIDURE-BL300 Series in which a negatively charged monomer is copolymerized, LIPIDURE-BL400 Series in which a positively charged monomer is copolymerized, LIPIDURE-BL700 Series in which a monomer having a hydrogen-bonding group is copolymerized, LIPIDURE-BL800 Series, LIPIDURE-BL1000 Series, LIPIDURE-BL1100 Series, LIPIDURE-BL1200 Series, and LIPIDURE-BL1300 Series in which a monomer having a hydrophobic group is copolymerized are preferable. Further, LIPIDURE (R)-BL103, LIPIDURE (R)-BL203, LIPIDURE (R)-BL206 (molecular weight of about 300000), LIPIDURE (R)-BL405, LIPIDURE (R)-BL502, LIPIDURE (R)-BL702, LIPIDURE (R)-BL802, LIPIDURE (R)-BL1002, LIPIDURE (R)-BL1201 (molecular weight of about 400000), or LIPIDURE (R)-BL1301 is more preferable.

The weight average molecular weight of the polymer having 2-methacryloyloxyethyl phosphorylcholine as the structural unit is not specifically limited, as long as desired performance is achieved. The weight average molecular weight and monomer ratio of the polymer having 2-methacryloyloxyethyl phosphorylcholine as the structural unit can be selected by those skilled in the art according to any purpose with reference to LIPIDURE-BL Series manufactured by NOF CORPORATION.

The lower limit of weight average molecular weight is, for example, 1000, preferably 2000, more preferably 5000, and most preferably 10000. The upper limit of weight average molecular weight is, for example, 2000000, preferably 1000000, further preferably 700000, further preferably 100000, and most preferably 70000. The weight average molecular weight can be measured by the method such as gel permeation chromatography.

The polymer having 2-methacryloyloxyethyl phosphorylcholine as the structural unit can be used singly, or any 2 or more can be used in combination. In addition to the polymer having 2-methacryloyloxyethyl phosphorylcholine as the structural unit, a surfactant such as a cationic surfactant or an anionic surfactant may also be added.

In the composition of the present invention, the order of addition of a polymer having 2-methacryloyloxyethyl phosphorylcholine as the structural unit and the TARC is not particularly limited, as long as the effects of the present invention are obtained.

### Concentration of Polymer Having 2-methacryloyloxyethyl Phosphorylcholine as Structural Unit

In the composition of the present invention, the concentration of the polymer having 2-methacryloyloxyethyl phosphorylcholine as the structural unit is not limited to the following examples, but is preferably 0.0001 mass % to 5 mass %, more preferably 0.001 mass % to 5 mass %, further preferably 0.01 mass % to 5 mass %, and most preferably 0.01 mass % to 1 mass %, in consideration of the stability of TARC, with respect to the composition.

### Storage Container

The composition of the present invention is preferably filled in a storage container. The material of the storage container is not specifically limited, as long as the effects of the present invention can be obtained, and sealing can be achieved, but at least part or all of the contact, area with the composition is plastic [such as an olefinic resin, a styrenic resin, an acrylic resin, a polyester resin, a polycarbonate resin, a fluorine resin, a chlorinated resin (such as polyvinyl chloride), a polyamide resin, a polyacetal resin, a polyphenylene ether resin (such as modified polyphenylene ether), polyarylate, polysulfone, a polyimide resin, a cellulose resin (such as cellulose acetate), and a hydrocarbon resin (including a halogen-substituted product)], a metal (such as aluminum), glass, or the like. Among them, in view of production, transportation, and storage of a calibration sample, plastic or glass is preferable, an olefin resin is preferable among plastics, and polypropylene is more preferable.

The storage container may be made of a single material or two or more materials but is preferably made of a single material. The storage container can include a container body and a cap. In this case, the container body and the cap may be made of different materials. In addition, the storage container, particularly the body part, preferably has transparency to the extent that the liquid content can be seen from the outside.

The form of the storage container may be either hard type or soft type, and examples thereof include ampoules, vials, soft bags, syringe-type containers, and glass bottles. The storage container is preferably in the form of a plastic eyedropper bottle, particularly in the form of a cylindrical eyedropper bottle, including a container body and a cap, for the ease of use and the stability of TARC. Further, the storage container is preferably in the form of a glass ampoule, or the form including a glass container body and a cap, particularly in the form including a cylindrical glass container and a rubber cap, for the ease of use and the stability of TARC.

### Composition

The composition of the present invention can be used as a calibration sample solution in the measurement of TARC. In this description, the calibration sample solution means a sample solution that is used for accurately measuring a substance to be measured and contains the substance to be measured at a certain concentration. Examples thereof include a standard substance, a calibrator, a control, and an internal standard substance. Examples of supply forms of the composition of the present invention include a solution state prepared in advance by mixing of a solvent with TARC and the polymer having 2-methacryloyloxyethyl phosphorylcholine as the structural unit.

In the TARC measurement method, “using a composition” means using a composition to measure TARC accurately. For example, it means using a liquid composition containing TARC as a calibration sample solution (such as a standard substance, a calibrator, a control, and an internal standard substance).

The pH of the composition of the present invention is, for example, 4.0 to 9.5, 5.0 to 9.0, 6.0 to 3.5, 6.5 to 8.0, or 7.0 to 8.0.

The pH can be adjusted by using a pH adjusting reagent well known to those skilled in the art, such as sodium hydroxide or hydrochloric acid.

The composition of the present invention may have any composition that is not particularly limited as long as it does not impair the effects of the present invention. When TARC is measured by immunoassay, the composition is suitable as long as it does not impair the effects of the present invention, and does not prevent all or part of the reactions constituting the assay system such as antigen-antibody reaction, labeling reaction for detection with biotin-avidin, and enzymatic reaction. Various components generally used in the immunoassay can be appropriately selected and used according to the purpose, including various buffers such as acetic acid, citric acid, phosphoric acid, PBS (phosphate buffered saline), HEPES, MES, Tris, glycine, boric acid, carbonic acid, and Good's buffers, components that promote antigen-antibody reactions (e.g., polymers such as polyethylene glycol and polyvinylpyrrolidone), glycoproteins and peptides (e.g., BSA and casein), amino acids, salts (e.g., sodium chloride and potassium chloride), saccharides (e.g., sucrose and cyclodextrin), and preservatives (e.g., sodium azide and ProClin300). PBS with a pH of 6.5 to 8.0 is preferably used.

In this description, “improvement in storage stability” or “improving the storage stability” means that most of TARC in a solution containing TARC is maintained without decomposing for a long period of time, without changing its structure, or without being adsorbed to a container, so that there is no significant difference between the initial value and the measured value after storage of the TARC in the solution.

More specifically, “improvement in storage stability” or “improving the storage stability” can mean, for example, that 80% or more of TARC in a solution containing TARC at a concentration of 10 pg/mL to 1 μg/mL is maintained without decomposing at 37° C. for 28 days, without changing its structure, or without being adsorbed to a container, so that the measured value of TARC in the solution after storage in a plastic container at 37° C. for 28 days is 80% or more of the initial value.

Further, “improvement in storage stability” or “improving the storage stability” can mean, for example, that 80% or more of TARC in a solution containing TARC at a concentration of 10 pg/mL to 1 μg/mL is maintained without decomposing at 4° C. for 28 days, without changing its structure, or without being adsorbed to a container, so that the measured value of TARC in the solution after storage in a plastic container at 4° C. for 28 days is 80% or more of the initial value.

Furthermore, “improvement in storage stability” or “improving the storage stability” can mean, for example, that 30% or more of TARC in a solution containing TARC at a concentration of 10 pg/mL to 1 μg/mL is maintained without decomposing at 10° C. for 12 hours, without changing its structure, or without being adsorbed to a container, so that the measured value of TARC in the solution after storage in a glass container at 10° C. for 12 hours is 30% or mere of the initial value.

### Biological Sample for Measurement of TARC

The biological sample for TARC measurement is not particularly limited as long as TARC can be measured, but blood, serum, or blood plasma is preferably used. The biological sample may be appropriately pretreated, as required. The biological sample is preferably a biological sample collected from a human.

### Kit for Measuring TARC

In the Kit for measuring TARC of the present invention, TARC can be measured conveniently and accurately by using the composition of the present invention. Examples of the Kit for measuring TARC can include a kit using an immunological technique. The Kit for measuring TARC of the present invention can contain a reagent for measuring the TARC concentration in a human body by an immunological technique. Examples of the immunological technique Include ELISA, enzyme immunoassay, surface plasmon resonance, latex agglutination immunoassay (LTIA), chemiluminescence immunoassay, electrochemiluminescence immunoassay, fluorescence antibody method, radioimmunoassay, Western blotting, immunochromatography, and high-performance liquid chromatography (HPLC).

The Kit for measuring TARC of the present invention can be used for selecting a treatment method or drug for atopic dermatitis, and for grasping the severity of atopic dermatitis when determining the effects of treatment.

The Kit for measuring TARC of the present invention can also include instructions for use and the like. The Kit for measuring TARC may contain optional components such as a buffer, a stabilizer, a sample diluent, a pH adjuster, and a reaction container.

### Method for Improving Storage Stability of TARC

The method for improving the storage stability of TARC of the present invention includes a step of contacting TARC with a solution containing a polymer having 2-methacryloyloxyethyl phosphorylcholine as the structural unit. After the polymer having 2-methacryloyloxyethyl phosphorylcholine as the structural unit is added to the solution, TARC may be added thereto, or after TARC is added to the solution, the polymer having 2-methacryloyloxyethyl phosphorylcholine as the structural unit, may be added thereto. The solution is preferably a buffer such as PBS, HEPES, MES, CHES, and Tris. PBS with a pH of 6.5 to 8.0 is more preferably used.

### Concentration of TARC

In the method for improving the storage stability of TARC of the present invention, the concentration of TARC contained in the solution is not limited to the following examples, but is preferably 10 pg/mL to 1 μg/mL, more preferably 50 pg/mL to 500 ng/mL, further preferably 100 pg/mL to 100 ng/mL, and most preferably 100 pg/mL to 50 ng/mL, in consideration of the stability of TARC.

### Polymer Having 2-methacryloyloxyethyl Phosphorylcholine as Structural Unit

In the method for improving the storage stability of TARC of the present invention, the polymer having methacryloyloxyethyl phosphorylcholine as the structural unit is not specifically limited, as long as it has 2-methacryloyloxyethyl phosphorylcholine as the structural monomer.

The polymer having 2-methacryloyloxyethyl phosphorylcholine as the structural unit may be a homopolymer of 2-methacryloyloxyethyl phosphorylcholine, or may be a copolymer of 2-methacryloyloxyethyl phosphorylcholine with a further monomer. The further monomer can have a hydrophobic side chain, a hydrophilic side chain, an anionic side chain, a cationic side chain, or a hydrogen-bonding side chain. The further monomer can also have both of a hydrophobic side chain and a hydrophilic side chain.

The constitutional ratio between 2-methacryloyloxyethyl phosphorylcholine and the further monomer may be variable depending on the structure of monomers to be used, but a content of 2-methacryloyloxyethyl phosphorylcholine is preferably 5 mol % or more.

The polymer having 2-methacryloyloxyethyl phosphorylcholine as the structural unit may be prepared according to a known synthesis method, or a commercial product such as LIPIDURE (R) Series available from NOF CORPORATION may also be used. LIPIDURE-BL Series is preferable, with LIPIDURE (R)-BL103, LIPIDURE (R)-BL203, LIPIDURE (R)-BL206 (molecular weight of about 300000), LIPIDURE (R)-BL405, LIPIDURE (R)-BL502, LIPIDURE (R)-BL702, LIPIDURE (R)-BL802, LIPIDURE (R)-BL1002, LIPIDURE (R)-BL1201 (molecular weight of about 400000), or LIPIDURE (R)-BL1301 being more preferable.

The weight average molecular weight of the polymer having 2-methacryloyloxyethyl phosphorylcholine as the structural unit is not specifically limited, as long as desired performance is achieved. The lower limit of such a molecular weight is, for example, 1000, preferably 2000, more preferably 5000, and most preferably 10000. The upper limit of such a molecular weight is, for example, 2000000, preferably 1000000, and further preferably 700000. The weight average molecular weight can be measured by the method such as gel permeation chromatography.

The polymer having 2-methacryloyloxyethyl phosphorylcholine as the structural unit may be prepared according to a known synthesis method as described in, for example, Japanese Patent Laid-Open No. 2004-169678. 2-methacryloyloxyethyl phosphorylcholine monomer is sold by, for example, NOF CORPORATION, and those skilled in the art can produce a polymer having required properties by polymerizing with any monomer. Alternatively, for example, LIPIDURE (R) Series sold by NOF CORPORATION may also be used, with LIPIDURE-BL Series sold as a diagnostic agent product being preferable, but is not limited thereto. LIPIDURE-BL Series are polymers in which various monomers are copolymerized with 2-methacryloyloxyethyl phosphorylcholine. LIPIDURE-BL100 Series in which a monomer having a hydrophilic group is copolymerized, LIPIDURE-BL200 Series in which a monomer having a hydrophobic group is copolymerized, LIPIDURE-BL300 Series in which a negatively charged monomer is copolymerized, LIPIDURE-BL400 Series in which a positively charged monomer is copolymerized, LIPIDURE-BL700 Series in which a monomer having a hydrogen-bonding group is copolymerized, LIPIDURE-BL900 Series, LIPIDURE-BL1000 Series, LIPIDURE-BL1100 Series, LIPIDURE-BL1200 Series, and LIPIDURE-BL1300 Series in which a monomer having a hydrophobic group is copolymerized, are preferable. Further, LIPIDURE (R)-BL103, LIPIDURE (R)-BL203, LIPIDURE (R)-BL206 (molecular weight of about 300000), LIPIDURE (R)-BL405, LIPIDURE (R)-BL502, LIPIDURE (R)-BL702, LIPIDURE (R)-BL802, LIPIDURE (R)-BL1002, LIPIDURE (R)-BL1201 (molecular weight of about 400000), or LIPIDURE (R)-BL1301 is more preferable.

### TARC Adsorption Inhibitor

The TARC adsorption inhibitor of the present invention includes a polymer having 2-methacryloyloxyethyl phosphorylcholine as the structural unit. The polymer having 2-methacryloyloxyethyl phosphorylcholine as the structural unit may be added to a storage container before contacting a solution containing TARC, or the polymer having 2-methacryloyloxyethyl phosphorylcholine as the structural unit may be added in advance as an adsorption inhibitor to a TARC-containing solution, and this solution may be added to a storage container.

Then, the present invention will be specifically described by way of Examples, but these do not limit the scope of the present invention. The product names, ingredient names, and distributors of the additives used in the Examples are as follows.
LIPIDURE-BL Series, available from NOF CORPORATION
ACETAMIN (R) 24 ingredient name: Coconut amine acetates, available from Kao Corporation
EMANON (R) 1112, ingredient name: polyethylene glycol monolaurate, available from Kao Corporation
Brij (R) 35, ingredient name: polyoxyethylene lauryl ether, available from KISHIDA CHEMICAL Co., Ltd.
Triton (R) X-100, ingredient name: polyethylene glycol mono-p-isooctylphenyl ether, available from KISHIDA CHEMICAL Co., Ltd.
MEGA-9, ingredient name: n-nonanoyl-N-methyl-D-glucamine, available from DOJINDO LABORATORIES
CHAPS ingredient name: 3-[(3-Cholamidopropyl) dimethylammonio]propanesulfonate, available from DOJINDO LABORATORIES

### EXAMPLES

### Example 1: Storage Stability Test Results in Glass Vials

The storage stability of TARC when stored in glass vials was tested. The test method and the evaluation method are as follows. A solution having the following constituents was used as a storage solution.
PBS (PH 7.2)
1 mass % BSA
Additives (concentration, product name, and type are described in Table 1)

### (1) Storage Condition

0.5 mL of a TARC liquid storage solution at each concentration was dispensed into glass containers and stored at 10° C. for 12 hours. A sample before dispensed into the glass container was also measured as a control. The TARC concentration was tested at 500 pg/ml., 2000 pg/mL, and 10000 pg/mL.

### (2) Measurement Method

Measurement was performed by latex immunonephelometry using two types of antibodies. The constituents of the reagents and the measurement method are shown below. Using first reagent and second reagent, measurement was performed using a HITACHI automatic analyzer.
First reagent

### 100 mM MOPS-NaOH (PH 7.5)

### 500 mM NaCl

### 0.5% BSA

Second reagent
   Anti-human TARC monoclonal antibody sensitized latex (two types)

### 5 mM MOPS-NaOH (PH 7.0)

Anti-Hunan TARC monoclonal antibodies were obtained using commercially available TARC antigens by methods well known to those skilled in the art. Examples of the commercially available TARC antigens included CCL17, thymus and activation regulated chemokine (Shenandoah Biotechnology, Inc.), CCL17/TARC, Human (LifeSpan Biosscience, Inc.), and Human TARC (CCL17) (Abeomics, Inc.). Further, a combination of monoclonal antibodies capable of a sandwich assay against the TARC antigen was selected by a method well known to those skilled in the art. The anti-human TARC monoclonal antibody sensitized latex was prepared with reference to the method described in Japanese Patent Laid-Open No. 2017-181377.

First, 120 μL of the first reagent was added to 2.4 μL of the TARC liquid storage solution at each concentration, and heated at 37° C. for 5 minutes. Then, 40 μL of the second reagent was added and stirred. Thereafter, absorbance changes for 5 minutes were measured at a dominant wavelength of 570 nm and a secondary wavelength of 800 nm. The absorbance changes measured were converted to TARC concentrations using a calibration curve obtained by measuring standard substances of known concentrations.

### (3) Calculation of TARC Residual Rate (%)

For the TARC concentration of each TARC liquid storage solution after storage at 10° C. for 12 hours, the TARC residual rate (%) was calculated using the following formula.

TARC residual rate (%)=TARC concentration of each TARC liquid storage solution after storage at 10° C. for 12 hours in a glass container (pg/mL)/TAPC concentration of TARC liquid storage solution before dispensed into a glass container (pg/mL)×100

(4) Table 1 shows the additives and concentration used under each condition, and Table 2 shows the evaluation results. The TARC residual rate (%) was calculated based on the average of three experiments.

TABLE 1
Concentration, product name, and type
of additives used in each condition

| | Additive | | |
|---|---|---|---|
| | Concen- | | |
| Condi- | tration | | Property of side chain in |
| tion | (mass %) | Product name | copolymerized monomer |
| 1 | — | None | None |
| 2 | 0.01% | Lipidure-BL206 | Hydrophobic side chain |
| 3 | 0.05% | | |
| 4 | 0.1% | | |
| 5 | 0.5% | | |
| 6 | 1.0% | | |
| 7 | 0.1% | Lipidure-BL103 | Hydrophilic side chain |
| 8 | 0.1% | Lipidure-BL405 | Anionic side chain |
| 9 | 0.1% | Lipidure-BL502 | Cationic side chain |
| 10 | 0.1% | Lipidure-BL702 | Hydrogen-bonding side chain |
| 11 | 0.1% | Lipidure-BL802 | Hydrophobic/hydrogen- |
| | | | bonding side chain |
| 12 | 0.1% | Lipidure-BL1002 | Hydrophobic side chain |
| 13 | 0.1% | Lipidure-BL1201 | Hydrophobic side chain |
| 14 | 0.1% | Lipidure-BL1301 | Hydrophobic side chain |
| 15 | 0.1% | Lipidure-BL206 | Lipidure-BL206 and cationic |
| | 0.01% | ACETAMIN 24 | surfactant in combination |

TABLE 2
Evaluation results (TARC residual rate
(%) when stored at 10° C. for 12 hours)

| | TARC | TARC | TARC |
|---|---|---|---|
| | 500 pg/mL | 2000 pg/mL | 10000 pg/mL |
| Condition | sample | sample | sample |
| 1 | 0.0 | 5.2 | 10.2 |
| 2 | 30.1 | 72.5 | 70.9 |
| 3 | 33.9 | 67.1 | 70.2 |
| 4 | 60.3 | 83.6 | 81.6 |
| 5 | 77.3 | 80.7 | 82.1 |
| 6 | 76.9 | 82.1 | 80.9 |
| 7 | 68.0 | 89.4 | 88.8 |
| 8 | 32.3 | 51.3 | 51.9 |
| 9 | 89.7 | 100.8 | 99.2 |
| 10 | 48.5 | 76.9 | 81.9 |
| 11 | 50.4 | 73.0 | 75.5 |
| 12 | 73.2 | 81.7 | 88.2 |
| 13 | 57.9 | 77.5 | 76.4 |
| 14 | 74.6 | 81.4 | 85.2 |
| 15 | 83.1 | 96.6 | 98.7 |

When the TARC concentration was 500 pg/mL, the residual, rate after 12 hours was 0.0% in condition 1 in which no polymer having 2-methacryloyloxyethyl phosphorylcholine as the structural unit was added/ presumably meaning that all TARC was adsorbed to the glass container, or the structure was changed. To the contrary, in conditions 2 and 3 in which 0.01 and 0.05 of LIPIDURE-BL206, which is the polymer having 2-methacryloyloxyethyl phosphorylcholine as the structural unit, was added, the residual rates were improved to 30.1 and 33.9%. Further, condition 4 in which 0.1% of LIPIDURE-BL206 was added, had the residual rate of 60.3%, and conditions 5 and 6 in which 0.5% and 1.0% of LIPIDURE-BL206 were added had the residual rates of 75% or more, which were significant improvement. Furthermore, TARC residual rates were significantly improved to 32.3% to 89.7%, with respect to condition 1, in conditions 7 to 14 in which 0.1% of compounds which are the polymers having 2-methacryloyloxyethyl phosphorylcholine as the structural unit and have various side chain structures was added.

When the TARC concentrations were 2000 pg/mL and 10000 pg/mL, TARC residual rates were similarly evaluated. The residual rates after 12 hours were 5.2% and 10.2% in condition 1 in which no polymer having 2-methacryloyloxyethyl phosphorylcholine as the structural unit was added, whereas the TARC residual rates were significantly improved to 51.3% to 100.8% in conditions 2 to 14 found in the present invention. It was demonstrated that a TARC residual rate is improved as long as the polymer having 2-methacryloyloxyethyl phosphorylcholine as the structural unit is used, regardless of properties of a side chain in a copolymerized monomer.

Furthermore, in condition 15 in which a cationic surfactant was used in combination with LIPIDURE-BL206 having 2-methacryloyloxyethyl phosphorylcholine as the structural unit, the TARC residual rate was significantly improved compared with condition 3. Further, the obtained results shows that the TARC residual rate was higher when a surfactant was used in combination with LIPIDURE-BL206 as compared with condition 4 in which LIPIDURE-BL206 at the same concentration was used alone.

It was demonstrated as above that coexistence of the polymer having 2-methacryloyloxyethyl phosphorylcholine as the structural unit and TARC improves the storage stability of TARC when stored in glass vials. This was considered because adsorption of TARC onto the wall surface of the container could be prevented by addition of the polymer having 2-methacryloyloxyethyl phosphorylcholine as the structural unit, or the structural changes of TARC during storage could be suppressed, in conditions 2 to 15. Further, it was suggested that the combination use of any surfactant with the polymer having 2-methacryloyloxyethyl phosphorylcholine as the structural unit can achieve further higher effect.

### Example 2: Storage Stability Test Results in Plastic Eyedropper Bottles

The storage stability of TARC when stored in plastic eyedropper bottles was tested. The test method and the evaluation method are as follows.

### (1) Storage Condition

0.5 mL of a TARC liquid storage solution at each concentration was dispensed into plastic eyedropper bottles and stored at 37° C. for 28 days or at 4° C. for 28 days. The TARC concentration was tested at 500 pg/mL, 2000 pg/mL, and 10000 pg/mL.

### (2) Measurement Method

The measurement was conducted by the same method as Example 1.

### (3) Calculation of TARC Residual Rate (%)

For the TARC concentration of each TARC liquid storage solution after storage at 37° C. for 28 days or at 4° C. for 28 days, the TARC residual rate (%) was calculated using the following formula.

TARC residual rate (%)=TARC concentration of each TARC liquid storage solution after storage at 37° C. for 28 days or at 4° C. for 28 days in an eyedropper bottle (pg/mL)/TARC concentration of TARC liquid storage solution immediately after preparation (pg/mL)×100

(4) Studies were conducted under the same conditions as Table 1, and Tables 3 and 4 showed the evaluation results. The TARC residual rate (%) was calculated based on the average of three experiments.

TABLE 3
Evaluation results (TARC residual rate
(%) when stored at 37° C. for 28 days)

| | TARC | TARC | TARC |
|---|---|---|---|
| | 500 pg/mL | 2000 pg/mL | 10000 pg/mL |
| Condition | sample | sample | sample |
| 1 | 47.2 | 86.4 | 87.3 |
| 2 | 94.8 | 97.4 | 91.5 |
| 3 | 88.5 | 98.0 | 93.9 |
| 4 | 92.6 | 94.9 | 95.5 |
| 5 | 84.9 | 94.6 | 91.6 |
| 6 | 90.2 | 88.8 | 88.3 |
| 7 | 90.3 | 94.5 | 94.9 |
| 8 | 90.7 | 100.6 | 97.9 |
| 9 | 89.0 | 95.8 | 93.3 |
| 10 | 96.1 | 97.2 | 95.3 |
| 11 | 88.2 | 97.5 | 96.8 |
| 12 | 91.2 | 91.9 | 100.7 |
| 13 | 87.9 | 102.4 | 94.9 |
| 14 | 92.5 | 92.6 | 92.5 |
| 15 | 83.9 | 93.0 | 93.7 |

When the TARC concentration was 500 pg/mL, the residual rate after 23 days was 47.2% in condition 1 in which no polymer having 2-methacryloyloxyethyl phosphorylcholine as the structural unit was added. To the contrary, in conditions 2 and 15 in which the polymer having 2-methacryloyloxyethyl phosphorylcholine as the structural unit was added, the residual rates as high values as 83.9 to 96.1 were maintained. Likewise, even when the TARC concentrations were 2000 pg/mL and 10000 pg/mL, the TARC residual rates were improved in conditions 2 to 15 in which the polymer having 2-methacryloyloxyethyl phosphorylcholine as the structural unit found in the present application was added, with respect to condition 1 in which no polymer having 2-methacryloyloxyethyl phosphorylcholine as the structural unit was added. This was considered because adsorption of TARC onto the wall surface of the container could be prevented by addition of the polymer having 2-methacryloyloxyethyl phosphorylcholine as the structural unit, or the structural changes of TARC during storage could be suppressed.

TABLE 4
Evaluation results (TARC residual rate
(%) when stored at 4° C. for 28 days)

| | TARC | TARC | TARC |
|---|---|---|---|
| | 500 pg/mL | 2000 pg/mL | 10000 pg/mL |
| Condition | sample | sample | sample |
| 1 | 65.7 | 86.6 | 92.6 |
| 2 | 88.6 | 98.4 | 97.4 |
| 3 | 88.9 | 97.8 | 100.3 |
| 4 | 93.8 | 100.3 | 99.8 |
| 5 | 88.5 | 95.6 | 102.0 |
| 6 | 87.4 | 96.7 | 100.7 |
| 7 | 95.3 | 97.6 | 100.9 |
| 8 | 96.6 | 97.0 | 101.4 |
| 9 | 82.1 | 97.7 | 99.1 |
| 10 | 92.2 | 96.5 | 99.6 |
| 11 | 81.1 | 95.4 | 101.1 |
| 12 | 87.3 | 97.2 | 100.9 |
| 13 | 83.1 | 99.0 | 101.0 |
| 14 | 95.7 | 95.8 | 101.1 |
| 15 | 88.4 | 94.3 | 97.8 |

When the TARC concentration was 500 pg/mL, the residual rate after 28 days was 65.7% in condition 1 in which no polymer having 2-methacryloyloxyethyl phosphorylcholine as the structural unit was added, which was a significantly low result. To the contrary, in conditions 2 and 15 in which the polymer having 2-methacryloyloxyethyl phosphorylcholine as the structural unit was added, the residual rates were as high values as 81.1 to 96.6%. Likewise, even when the TARC concentrations were 2000 pg/mL and 10000 pg/mL, TARC residual rates were improved in conditions 2 to 15 in which the polymer having 2-methacryloyloxyethyl phosphorylcholine as the structural unit found in the present application was added, with respect to condition 1 in which no polymer having 2-methacryloyloxyethyl phosphorylcholine as the structural unit was added. This was considered because adsorption of TARC onto the wall surface of the container could be prevented by addition of the polymer having 2-methacryloyloxyethyl phosphorylcholine as the structural unit, or the structural changes of TARC during storage could be suppressed in conditions 2 to 15.

### Comparative Example 1: Storage Stability Test Results in Glass Vials

The storage stability of TARC when stored in glass vials was tested. A solution having the following constituents was used as a storage solution.
PBS (PH 7.2)
1 mass % BSA
Surfactant (concentration, product name, and type are described in Table 5)

The experiment was performed in the same procedure as Example 1, except that surfactants were used as the additive and the storage time was 6 hours.

TABLE 5
Concentration, product name, and type
of additives used in each condition

| | Surfactant | | | |
|---|---|---|---|---|
| | Concen- | | | |
| Condi- | tration | | | |
| tion | (mass %) | Product name | Type | |
| 1 | — | None | None | |
| 16 | 0.01% | EMANON 1112 | Nonionic | Polyethylene |
| | | | | glycol fatty acid |
| | | | | ester type |
| 17 | 0.01% | Briji35 | Nonionic | Alcohol type |
| 18 | 0.01% | TritonX-100 | Nonionic | Alkyl phenol type |
| 19 | 0.01% | MEGA-9 | Nonionic | Sugar amide type |
| 20 | 0.01% | CHAPS | Amphoteric | Bile acid type |

TABLE 6
Evaluation results (TARC residual rate
(%) when stored at 10° C. for 6 hours)

| | TARC | TARC | TARC |
|---|---|---|---|
| | 500 pg/mL | 2000 pg/mL | 10000 pg/mL |
| Condition | sample | sample | sample |
| 1 | 4.4 | 42.7 | 65.4 |
| 16 | 4.1 | 49.9 | 69.2 |
| 17 | 1.4 | 51.9 | 74.5 |
| 18 | 7.8 | 52.5 | 72.8 |
| 19 | 0.6 | 50.9 | 69.4 |
| 20 | 3.9 | 48.6 | 69.2 |

When the TARC concentration was 500 pg/mL, the residual rate after 6 hours was 4.4% in condition 1 in which no surfactant was added, presumably meaning that most of TARC was adsorbed onto the glass container, or the structure was changed. To the contrary, in conditions 16 to 20 in which the nonionic surfactants or the amphoteric surfactant having specific structure was added, the residual rates after 6 hours were 0.6% to 7.8%, which were not improved as compared with condition 1 in which no surfactant was added. Likewise, when the TARC concentrations were 2000 pg/mL and 10000 pg/mL, in conditions 16 to 20 in which the nonionic surfactants or the amphoteric surfactant having specific structure was added, the TARC residual rates were net improved as compared with condition 1 in which no surfactant was added.

Accordingly, it was considered that the polymer having 2-methacryloyloxyethyl phosphorylcholine as the structural unit has better TARC storage stability improving effect than the nonionic surfactants and amphoteric surfactant having specific structures used in Comparative Example 1. In particularly condition 20, the compound described in Patent Literature 2 was added, however no effect of improving storage stability was found at all the TARC concentrations studied.

### INDUSTRIAL APPLICABILITY

The present invention can provide a TARC-containing composition with high storage stability, particularly, a TARC-containing calibration sample.

1. A composition comprising:
TARC (Thymus and activation-regulated chemokine); and
a polymer having 2-methacryloyloxyethyl phosphorylcholine as a structural unit, and being in liquid form.

2. The composition according to claim 1, wherein the composition is a calibration sample solution for measuring TARC.

3. The composition according to claim 1, wherein the composition is filled in a storage container.

4. The composition according to claim 3, wherein the storage container is plastic or glass.

5. The composition according to claim 1, wherein the TARC concentration is 10 pg/mL to 1 μg/mL with respect to the composition.

6. The composition according to claim 1, wherein a concentration of the polymer is 0.001 mass % to 5 mass % with respect to the composition.

7. The composition according to claim 1, wherein the polymer has a weight average molecular weight of 1000 to 2000000.

8. The composition according to claim 1, wherein when the TARC concentration is 500 pg/mL with respect to the composition, the TARC residual rate is 80% or more after storage at 37° C. for 28 days in a plastic container.

9. The composition according to claim 1, wherein when the TARC concentration is 500 pg/mL with respect to the composition, the TARC residual rate is 80% or more after storage at 4° C. for 28 days in a plastic container.

10. The composition according to claim 1,
wherein when the TARC concentration is 500 pg/mL with respect to the composition, the TARC residual rate is 30% or more after storage at 10° C. for 12 hours in a glass container.

11. The composition according to claim 1,
wherein the polymer has a weight average molecular weight of 10000 to 70000.

12. A method for measuring TARC, wherein the method uses the composition according to claim 1.

13. A kit for measuring TARC, wherein the kit comprises the composition according to claim 1.

14. A method for improving storage stability of TARC, comprising a step of contacting TARC with a solution containing a polymer having 2-methacryloyloxyethyl phosphorylcholine as a structural unit.

15. The method for improving the storage stability of TARC according to claim 14, wherein the TARC concentration is adjusted so as to be 10 pg/mL to 1 μg/mL with respect to the solution.

16. The method for improving the storage stability of TARC according to claim 14, wherein a concentration of the polymer is 0.001 mass % to 5 mass % with respect to the solution.

17. The method for improving the storage stability of TARC according to claim 14, wherein the polymer has a weight average molecular weight of 10000 to 70000.

18. An adsorption inhibitor for preventing adsorption of TARC in a solution containing TARC onto a container, comprising a polymer having 2-methacryloyloxyethyl phosphorylcholine as a structural unit, as active component.

19. The adsorption inhibitor according to claim 18, wherein a TARC concentration in the solution containing TARC is 10 pg/mL to 1 μg/mL with respect to the solution.

20. The adsorption inhibitor according to claim 18, wherein the polymer has a weight average molecular weight of 10000 to 70000.
